(19) United States
(12) Patent Application Publication
(10) Pub. No.: US 2007/0122363 A1
(43) Pub. Date: May 31, 2007

(54) CORPOREAL DELIVERY OF CAROTENOIDS

(76) Inventors: Martin S. Giniger, New York, NY (US); Matthew S. Spaid, New York, NY (US); Brian Olson, New York, NY (US)

Correspondence Address: BERENBAUM, WEINSHIENK & EASON, P.C, 370 17TH STREET, SUITE 4800, DENVER, CO, 80202, US

(21) Appl. No.: 11/563,617

(22) Filed: Nov. 27, 2006

Related U.S. Application Data
(60) Provisional application No. 60/739,635, filed on Nov. 26, 2005.

(51) Int. Cl. A61K 8/97; A61K 36/82; A61K 36/00; A61K 36/16; A61K 36/28; A61K 36/87

(52) U.S. Cl. 424/58; 424/725; 424/729; 424/764; 424/752; 424/766

(57) ABSTRACT

A method for delivery of a phytochemical supplement to a corporeal body. The method of delivery comprises providing a phytochemical supplement in an orally acceptable composition and applying the orally acceptable composition including the phytochemical supplement in contact with a feature of the oral cavity of a corporeal body. An oral care composition comprising a phytochemical supplement for one or more of ingestion or trans-mucosal absorption and a method for making the same are also disclosed.

### CROSS REFERENCE TO RELATED APPLICATIONS

The present invention claims the benefit of and priority from the prior-filed U.S. Provisional Patent Application, No. 60/739,635; filed Nov. 26, 2005, entitled “Apple Extract in Oral Care”; the subject matter of which hereby being specifically incorporated herein by reference for all that it discloses and teaches.

### BACKGROUND

1. Field of Invention

The disclosure here relates to carotenoids and/or other supplemental phytochemicals or phytonutrients and delivery thereof to a corporeal body. Example methods of formulation and use, and descriptions of compositions of matter are disclosed.

2. The Prior Art

Many phytochemicals that include or may be derived from fruit or vegetable extracts such as those available from apples and tomatoes, inter alia, are believed to have a beneficial effect on health or play an active role in the amelioration of disease, e.g., they may have medicinal value. A variety of mechanisms may be responsible for any medicinal or otherwise potentially physically beneficial properties; however, none so far elucidated may be conclusive of any or all physically beneficial properties nor of the exact mechanisms.

Carotenoids are a class of phytochemicals that are widely present in nature, e.g. in leaves and fruits of plants, vegetables, and algae. Carotenoids are also distributed widely in animals that ingest these plants, and are also contained in shells of Crustacea such as crab, shrimp, and crawfish, in salmon muscles, in echinoid, and in eggs of Theragra chalcogramma, inter alia. Some specific non-limitative examples of carotenoids include lycopene, which is abundantly contained in tomatoes, beta-carotene, which is abundantly contained in carrots, alpha-carotene, astaxanthin, cryptoxanthin, zeaxanthin, lutein, canthaxanthin, fucoxanthin, and vitamin A. It has been suggested that carotenoids may protect plants from active oxygen generated during the photosynthesis of chlorophyll in the plant or alga cells. Moreover, though the reasons have not yet been clarified, it is believed that carotenoids may also protect animals or other living organisms from adverse effects caused by active oxygen species, e.g., free radicals, and the like. It has still further been reported that carotenoids may also have desirable functions in humans who ingest foods containing these ingredients.

Note, some of the more specific asserted benefits include reports that supplemental dietary carotenoids may inhibit the development of cancer; e.g., in some instances that lycopene may antagonize the progression of colon cancer. Carotenoids have also been significantly implicated in cardiovascular health. Furthermore, lycopene analogs may decrease a risk of age-related retinal macular degeneration associated with diabetes. Additionally, beta carotene may change ocular dysfunction for the better, and chronic use of small amounts of supplementary carotenoids may prevent cataracts.

It is believed that many of the effects described above may be caused by antioxidative activities of carotenoids, in particular, removing active oxygen and/or free radicals generated in living organisms. Therefore, the functions of carotenoids as described above may be very important for living organisms. As a consequence, increasing the uptake of carotenoids would likely be beneficial for living organisms.

Furthermore, long-term inadequate intake of carotenoids has been thought to be associated with chronic disease, including heart disease and various cancers. One mechanism for this carotenoid-disease relationship may be in association with free radicals. It has been suggested that diets low in beta-carotene and carotenoids may increase the body's susceptibility to damage from free radicals. As a result, over the long term, beta-carotene-deficient diets may increase tissue damage from free radical activity and increase risk of chronic diseases like heart disease and cancers. Diets low in beta carotene may reduce the effectiveness of the immune system and lead to an increased risk of cancer and heart disease. Older persons with type II diabetes may have a significant age-related decline in blood levels of carotenoids, irrespective of dietary intake. Symptoms of a beta-carotene deficiency mimic those of a vitamin A deficiency: dry skin, night blindness, susceptibility to infection. Acute carotenoid deficiency has not been seen with large consequence in the western world, although in some African sub-populations acute deficiencies have been reported to be as high as 75% or greater. However, in America, where apples, tomatoes, carrots, and the like are plentiful, it has been rare to find persons devoid of any ingestion of carotenoids. Instead a more common situation is under-exposure over a long term which may create a chronic deficiency in old age, possibly leading to many common ill effects that may be life threatening.

Although complete carotenoid deficiency is unlikely in developed nations where apples, tomatoes, carrots, and the like are plentiful, the mean daily intake of most Europeans and Americans has been thought lower than ideal, and over the course of years may be directly linked with many important diseases. It has been estimated that more than 50% of US adults consume less than a recommended 6 to 8 mg of carotenoids on a mean daily basis. Furthermore, tomatoes, carrots and apples are often eaten raw, and thus provide much less bio-available carotenoids. Some studies have shown that cooking vegetables and fruits improves the availability of carotenoids. For example, the availability of lycopene from either tomato sauce or apple sauce has been found to be increased when processed at high temperatures causing maximal destruction of cell walls and leakage of cell contents. As a result, it appears that much more lycopene and/or vitamin A is available for absorption by a corporeal body, thus, bio-available, from apple extracts than from a raw crisp apple.

The recommended amount of carotenoid intake varies by age and other factors. No recommended daily allowance (RDA, also known by the more recent appellation of Reference Daily Intake, RDI) has yet been established for carotenoids as a general class, but RDAs have been set for vitamin A, which may be derived from beta carotene. For example, the RDA of Vitamin A in adults is between about 600 and 900 μg/day (which may be equivalently derived from between about 7 and about 11 mg of beta carotene, about 12 times the vitamin A amount). Furthermore, it has been reported that that the levels of carotenoid intake associated with lowest risk of chronic disease may be even higher, up to between about 25 and 50 mg of beta carotene per day. In sharp contrast, an average “bio-available” intake of beta-carotene and lycopene in the United States and Europe may typically be only on the order of about one tenth or about one twentieth or less than the desired amount per day (e.g., at about 2 mg of beta carotene per day). Moreover the trend towards eating raw vegetables as a healthier alternative to cooked vegetables further reduces the population's exposure to usable, i.e., bio-available, carotenoids derived from foods. Consequently a large gap appears to exist between mean bio-available intake, the RDA amount, and the amount needed to reduce the risk of disease.

Thus, it was concluded that there could likely be beneficial and useful consequences to development of new carotenoid delivery systems and/or extractions of carotenoids from raw sources. In some views this would include increasing the bioavailability of carotenoids. Considering the potential for possibly lowering the incidence of certain cancers, heart disease and/or blindness, the present developments may represent a significant advancement in delivery of carotenoids and/or other phytochemical supplements.

### SUMMARY

The present invention is directed generally to formulations of orally acceptable compositions containing a phytochemical supplement, and to methods of making and use thereof. The phytochemical supplement may be an apple extract or may be a carotenoid.

The detailed description set forth hereinbelow is intended as a description of a variety of exemplary oral care products including phytochemical, carotenoid and/or apple extract compositions in some cases with beta-carotene or lycopene or similar carotenoids. The description sets forth features and/or operations for preparing and using the oral care compositions according hereto. It is to be understood, however, that the same or equivalent functions and ingredients incorporated in such compositions may be accomplished by different embodiments that are nevertheless also intended to be and are encompassed within the spirit and scope of the present invention.

### DETAILED DESCRIPTION

The presently-described developments involve carotenoid or other phytochemical delivery to a corporeal body. In many implementations, this involves oral care compositions, typically of a daily-use form, infused with a carotenoid or other phytochemical supplement, often including one or more or a combination of apple extract derived from cooked dried apples and/or another bio-available carotenoid supplement such as vitamin A, e.g., in a form such as beta carotene or lycopene. Such compositions are an effective way to increase intake of available vitamin A or other carotenoids or phytochemicals on a reliable basis; particularly simply and/or on large scales, e.g., potentially population-wide. It may be that by providing a reliable everyday source of bio-available carotenoids, particularly in an oral care form, such oral care products may be quite useful as they can contribute to achieving an optimal daily dose. Such products could contain a controlled level of carotenoids that would not cause toxicity if accidentally ingested, but may leave a significant amount of residual active agent deposited on the oral tissues, after expectoration. In some situations, a predictable residual amount may be in the range of about 1% to about 5% of the total amount initially applied into the oral cavity.

Carotenoids derived through an extraction method described below, e.g., from dried apples, can be added to oral care products at a substantially safe, non-toxic level of about 50 mg per unit dose with an expected oral tissue retainment with subsequent dietary systemic benefit of about 0.5 to about 2.5 mg per use. An oral care product of such a type, used twice daily, may provide an increased systemic intake of from about 1 to 5 mg daily in human usage, and such increased exposure could have long term significant health benefits for the individual and for the population at large. A desirable dose of about 50 mg of carotenoid supplementation per day is based on the Physicians Health Study conducted by the National Institutes of Health (NIH) and Harvard University that compared results in about 22,000 male physicians consuming in respective substantially equal groups either 50 mg of beta-carotene daily or a placebo (sugar pill). Results of that study found asserted benefits and no adverse health effects at that level.

Note, although oral care products are not typically intended for direct ingestion, a residual amount nevertheless does remain, sometimes intentionally (e.g., for long-term protection or release of effective ingredients) in the oral cavity even after expectoration. In some situations, as much as about 5% of residual toothpaste, for example, may remain on teeth after use. Such a residual amount may be swallowed or may otherwise be ingested, as absorbed through a mucosal lining, over the course of a day. And, when such an oral care product contains a desirable supplement, such as a carotenoid or other phytochemical, this will provide a simple, reliable delivery mechanism systemically or locally or both.

Thus, many implementations hereof are directed to the use of natural extracts as from fruits or vegetables, alone or in optional combination with specific carotenoids such as lycopene or beta carotene as one or more effective ingredients in oral care products. This may be useful to conveniently and predictably increase exposure to bio-available carotenoids that can help an individual achieve exposure to the optimal level of dietary carotenoids to lower risk of many local and/or systemic diseases. One implementation is in a mouth rinse. Another implementation is in a toothpaste. Yet another is in a denture adhesive. Of particular note in or from an apple extract example are the beta-carotenoids and vitamins such as vitamin A and the antioxidant properties thereof. Such compositions may provide for a naturally antimicrobial effect which can thus provide a naturally preservative effect.

Of particular note may be the provision of carotenoids, which as described above, are often found deficient in contemporary diets. For example the provision of vitamin A particularly in an oral care product can provide a surplus of vitamin A which may be slowly released into the physical system relatively continuously over a period, such as over the period of a day or a substantial part thereof. Such a provision over an extended period can provide for a constant sufficient amount of the vitamin within the physical system even as against the continual removal thereof by the excretory system, inter alia. Note further that vitamin A is a recyclable vitamin inasmuch as it remains in the system, and so long as a supply is provided, this recyclability may be improved. Suffering from vitamin A deficiency can thereby be reduced.

The mechanism of delivery of the beneficial components of an apple extract or other carotenoid or phytochemical ingredient from an oral care composition may be via absorption in and/or through the oral tissues, or may more readily be by the continual swallowing of residual amounts in the oral cavity. Thus, if applied as part of a tooth paste or like oral care product, such product may remain in residual amounts within the oral cavity for an extended period such as the period of a day or a substantial portion thereof.

As described in further detail below, the ingredient of an apple extract into an oral care composition may be in the form of an apple extract directly, or it may be in conjunction with other ingredients such as flavorings which may include apple with cinnamon, sour apple or apple mint; in any of which latter cases, an additional flavor (apple, cinnamon, sour, or mint, inter alia) not from extract may optionally be added. Note, apple extracts useful here may be organic and/or kosher and will typically be substantially safe, naturally.

The major contributors of carotenoids to the human diet vary by specific type. Alpha-carotene is primarily derived from carrots consumed as a single food or as an ingredient in multi-component foods. Carrots, apples, particularly yellow apples, cantaloupe, and broccoli are the main sources of beta-carotene (also known as a precursor of vitamin A). Orange juices and blends, oranges, and tangerines are important contributors of beta-cryptoxanthin. Apples and tomato products consumed as single foods or as ingredients in multi-component foods provide most dietary lycopene. Contributors of lutein and zeaxanthin include collard, mustard, or turnip greens, spinach, and broccoli. Of all the carotenoids, lycopene and beta carotene have recently been the most implicated in having significant effects on systemic health.

Anti-oxidants and other phytochemicals suitable for human ingestion include Vitamin E (e.g., tocopherol), Vitamin A (retinol), Vitamin C (ascorbic acid), carotenoids, echinacoside and caffeoyl derivatives, oligomeric proanthocyanidins or proanthanols (e.g., grape seed extract), silymarin (e.g., milk thistle extract, Silybum marianum), ginkgo biloba, green tea polyphenols, and the like, and mixtures thereof. Indeed, any pharmaceutically acceptable compounds suitable for administration orally in oral care products may be useful herein, either alone or in any combination with carotenoids. In many implementations, the phytochemical or anti-oxidant component may include an extract of yellow unpeeled apples or an isolated stable carotenoid.

Carotenoids which may be useful individually or in combination herein include, for example, beta-carotene, canthaxanthin, zeaxanthin, lycopene, lutein, crocetin, capsanthin, and mixtures thereof. To date, about 600 different kinds of carotenoids have been isolated from fruits and vegetables and any or all of these varieties of carotenoids may foreseeably be useful in implementations hereof. These include, but are not limited to: lycopene (gamma, gamma-carotene), beta-carotene (beta, beta-carotene), alpha-carotene ((6′R)-beta, epsilon-carotene), beta-cryptoxanthin ((3R)-beta, beta-caroten-3-ol), zeaxanthin ((3R,3′R)-beta, beta carotene-3,3′-diol), lutein (“xanthophyll”, (3R,3′R,6′R)-beta, epsilon-carotene-3,3′-diol), neoxanthin ((3S,5R,6R,3′S,5′R,6′S)-5′,6′-epoxy-6,7-didehydro-5,6,S′,6′-tetrahydro-beta, beta-carotene-3,5,3′-triol), violaxanthin ((3S,5R,6R,3′S,5′R,6′S)-5,6,5′,6′-diepoxy-5,6,5′,6′-tetrahydro-beta, beta-carotene-3,3′-diol), fucoxanthin ((3S,5R,6S,3′S,5′R,6′R)-5,6-epoxy-3,3′,5′-trihydroxy-6′,7′-didehydro-5,6,7,8,5′,6′-hexahydro-beta, beta-caroten-8-one 3′-acetate), canthaxanthin (beta, beta-carotene-4,4′-dione), and astaxanthin ((3S,3′S)-3,3′-dihydroxy-beta, beta-carotene-4,4′-dione).

A carotenoid component, most preferably containing an extract of a fruit containing both beta carotene and lycopene, would typically be present in an amount from about 1 to about 10 weight percent, and preferably from about 5 to about 10 weight percent in an oral care or orally applied composition hereof. Any natural or artificially manufactured carotenoid or phytochemical may be used, whether it be in lyophilized, powdered, crystalline, liquid tincture or other physical and/or chemical form.

Pure or substantially pure forms of carotenoids or other phytochemicals are useful herein whether naturally derived or synthesized. However; many implementations will feature natural forms, particularly in fruit or vegetable extract forms, because the fruits and vegetables will often have or retain, even in extract form, a number of useful properties. As an example, apple extracts are a good source of potassium to help lower blood pressure. Raw apples are also a great source of soluble fibers such as pectin. This property helps lower blood cholesterol levels and blunts the blood sugar surges after a meal. However the fiber content of plant cell walls may also prevent many other nutrients from being bio-available and absorbable when a raw apple is eaten. Conversely, if the apple is extracted, juiced, cooked, blanched or chemically treated, it is possible to unlock many hidden nutritive elements found in different apple varieties.

Apple cell fluid, once obtained through the removal or breakdown of cell walls, contains a good supply of vitamin C, as well as a number of health-promoting phytochemicals, such as carotenoids. The skin of an apple has about 8 to 10 times more carotenoids than the pulp. Different varieties of apples contain different pigments in their skins. Yellow apples, such as golden delicious, contain a high level of carotenoids. Green apples, such as Granny Smiths, contain chlorophyll, while red apples such as red delicious contain anthocyanins. All apples, and especially whole yellow apples, including the skins, are very rich in carotenoids that give fruit its antioxidant properties. Antioxidants are believed to counteract the damaging effects of free radicals, which have been associated with Alzheimer's disease, heart disease and many cancers. In a recent study, eating the equivalent of one apple a day reduced the risk of cancer of the mouth by 21%, colon cancer 20%, breast cancer 18%, and prostate cancer 9%. Human studies have linked apple extract consumption with a reduced risk of asthma, lung cancer, diabetes, and a 20 percent lower risk of cardiovascular disease. The carotenoids in apples, with their strong antioxidant activity, can lower cholesterol levels and protect LDL cholesterol from being oxidized, thereby reducing the risk of heart disease.

An apple extract containing active, bio-available carotenoids is included in many compositions hereof, and this preferably is an extract of yellow whole apples, including skins, although a mixture of one or more or all of yellow, green and red apples may also be quite healthful and useful herein. Extraction according hereto may be of unpeeled apples using a blanching pre-treatment and an enzymatic degradation of cell walls. The carotenoid component extracted from apples, when used, may typically be present in an amount from about 1 to 50 weight percent, more preferably from about 3 to 20 weight percent, and most preferably from about 5 to 10 weight percent of the composition. Toothpaste and denture care formulations of the composition that have a higher degree of substantivity and tissue coating ability, however, will typically include the carotenoid component in an amount from about 0.5 to 15 weight percent, preferably from about 1 to 10 weight percent. Mouth rinse compositions with lower oral tissue binding activity will usually have larger amounts included to yield an equivalent level of ingested or absorbed carotenoid benefit.

Bio-available carotenoids used herewith may be produced through a suitable extraction process that results in a commercially viable high yield that is stable under room temperature or refrigeration conditions. A final oral care product having a high quality of bio-available carotenoids may be dependent to a degree on the extraction process. Care should be taken during the extraction process as carotenoids may exhibit some instability in the presence of light and oxygen. Carotenoids also tend to be water insoluble unless bound to proteins, so care should be taken not to dissociate the two. Furthermore a method should be selected that avoids large pH swings. Extreme acid and alkaline conditions can also be detrimental to certain carotenoids. Furthermore, some enzymes found in fruits and vegetables rich in carotenoids, especially the lipoxygenase enzyme, may partially destroy the carotenoids. Thus, if the carotenoids are not protected through various techniques during extraction, then degradation, isomerization and/or oxidation may occur.

A simple, mostly mechanical method of apple extraction involves either heating un-peeled apple slices or heating these slices under heat and pressure, to release the phytochemicals or phytonutrients contained within the cell fluids trapped within the cell walls of apple cells. After softening, the apple slices can be ground up using a variety of methods including homogenization, shearing in a lab or commercial blender, or forced pressing through a sieve, although a varieties of other methods can also be used. The extract can then be further purified by reaction with a solvent, and passed through one or more filters to remove debris and trap the fraction that contains the useful carotenoids.

Chemical methods of carotenoid extraction typically have concentrated on the use of one or a combination of solvents. D-limonene has been used for extraction for orange peels. Petroleum ether:acetone (50:50 v/v) mixtures have been used as solvents. A rapid extraction method (15 minutes) has been used for obtaining lycopene and b-carotene from tomato paste and pink grapefruit homogenates using hexane:acetone:methanol (50:25:25 v/v/v) followed by addition of water to separate the solution into polar and non-polar layers. Lycopene was collected from the upper non-polar phase while the rest stayed in the polar phase. A hexane:acetone:methanol (80:10:10 v/v/v) ternary system in the AOAC method for xanthophylls was modified to hexane: acetone: methanol (70:20:10 v/v/v) to increase the solubility of carotenoids since the AOAC method was inadequate for dissolving polar xanthophylls. Fractionation of thylakoid membranes at low temperatures and non-toxicity was achieved by solubilizing the membranes with low concentrations of glycosidic surfactants (dodecylmaltoside) and fractionating by sucrose gradient ultracentrifugation with flat-bed isoelectric focusing (IEF).

Individual carotenoids can be identified and separated by reversed phase HPLC in a chromasil C18 column. Extraction of a-crocin from saffron may be achieved thusly by using only cold water, since the pigment is water soluble. Methanol:THF (50:50 v/v) may similarly be used, along with sodium-sulfate as a drying agent and magnesium-carbonate as a neutralizing agent. Extraction may be carried out in dark conditions at 4° C. to minimize carotenoid loss. Dichloromethane/methanol (2:1, v/v) has also been used for the vegetable carotenoid extraction.

Besides the common solvent extraction, carotenoids have been extracted from samples by using supercritical carbon dioxide, CO₂. In this technique, liquid CO₂ may be pumped through the extraction vessel and extracted carotenoids collected in the depressurized stream of supercritical CO₂. Yields may be increased by increasing the volume of supercritical CO₂, 80% being a reported maximum.

Enzyme extraction of carotenoids may provide a desirable approach. In some implementations, this may involve isolation and lysis of the chromoplasts followed by gel filtration, for isolation and purification of carotenoproteins from apples or carrots. For example, a method of enzyme extraction may use pectinase and cellulase enzymes to disrupt the cell wall of samples and release the carotenoids in the cell fluids. These carotenoids remain in their natural state still bound with proteins. Carotenoids bind with proteins through covalent bonding or weak interactions depending on their structure. This bonded structure prevents carotenoid oxidation and also affects color. Using this method, the carotenoids will be water soluble, which is preferable for oral care formulations. Furthermore, the enzyme extraction method is preferable over using methanol or acetone extraction methods that tend to dissociate the carotenoids from the proteins and cause water insolubility and ease of oxidation. Although enzyme extraction provides more stable carotenoids, it also preserves their characteristics during storage. This is a desirable objective because these products have the highest ability to make the final product attractive and acceptable. Degradation of carotenoids not only affects the attractive color of foods but also their nutritive value and flavor. Hence an enzyme extraction method using pectinase and cellulase enzymes to disrupt the cell walls may be preferred in many implementations, as these carotenoids are stable for up to a half (½) year. Furthermore, it appears that activity and stability of carotenoids can be further doubled if samples of fruits or vegetables are first pre-blanched in hot water. It may be that such pre-blanching destroys undesirable enzymes and/or other molecules that would otherwise have a destabilizing or oxidative effect on final isolated carotenoid powder over time.

The following methods may be used to recover high amounts of active carotenoids that remain stable under refrigeration and room temperature for the greatest number of days. Using the following methods, an oral care product containing active bio-available carotenoids which in some instances can be useful for up to about one year.

In some processes, blanching treatments may be performed prior to or as a preliminary part of the extraction process. In a first example, yellow apples may be selected to be used. These may then be cored, but in many instances preferably not peeled. These can then be sliced or diced or otherwise reduced in size and may then optionally be dried in a dehydrator. If not previously diced, they may be further reduced after drying. Otherwise, the recovered dessicated samples or, if not dried, the fresh cut samples, may then be wrapped in cheese cloth or the like and immersed in a water bath containing in some examples, distilled water at 100° C. for 2 minutes or the like to inactivate possible peroxidase activity. Residual water may then be poured off and the samples then homogenized in a blender such as a Waring blender for period such as about 2 minutes. Such a blanching prior to extraction may, in some cases, have a significant effect on the carotenoid stability and length of time that storage is possible under a range of temperature conditions. Using the above method, carotenoids may remain stable for as long as about one (1) year when stored at about 4° C. in a refrigerator.

Carotenoid extraction using enzymes may then be achieved in some examples. A fruit sample, preferably the pretreated and homogenized yellow unpeeled apples from the previously-described process, may then be placed in a large beaker. Commercial pectinase and cellulase enzymes may then be added. Samples may then be stirred using in some examples, a Corning PC 351 brand magnetic stirrer, at medium speed for an extended period, as, for example, about 24 hours at room temperature. About 2.5 ml of pectinase and 2 g of cellulase maybe used per an amount of about 100 g of resultant fruit sample in about 200 ml of distilled water. After about 24 hours enzyme treatment, in this example, a celite bed may be prepared on a porcelain Buchner funnel on #1 Whatman filter paper. Approximately 2 g of celite may be added to the enzyme treated fruit mixture and allowed to stand for about 10 minutes. The mixture may then be vacuum filtered through the celite bed. The first filtrate will be the water soluble carotenoid extract. Washing with water may be repeated until the celite bed returns to its original gray color. The watery colored fraction can be evaporated by using a rotary evaporator such as a Buchi EL 130 brand rotary evaporator at about 50° C. The remaining powdered residue will be a carotenoid rich extract of apples. Note, the product may ultimately be lyophilized, powdered, crystalline, liquid tincture or in other physical and/or chemical form.

In an alternative carotenoid extraction process, acetone or ethanol solvents may be used. The fruit sample, preferably pretreated and homogenized apples as described above, may be placed in a large beaker. These may then be weighed and blended with about 10 volumes of cold (i.e. −10° C.) acetone for about 1 minute in a blender such as a Waring Blender. After filtration such as on a Buchner funnel with suction, the material may be spread over a large surface, and allowed to dry at room temperature. The extract sample may then be passed through a sieve of, for example, about 80 mesh and stored in a vacuum desiccator at about 4° C. The acetone powder may then be suspended in about 10 volumes of 0.02 M potassium phosphate buffer, pH 7.0, stirred for 10 minutes at 0° C. Distilled water may then be added to the acetone extract until precipitation occurs. The acetone may then be evaporated by using a Buchi EL 130 brand rotary evaporator at 50° C. The remaining powdered residue will be a carotenoid rich extract of apples. Ethanol (95%) can be substituted for acetone in this method with substantially equivalent yields and results obtained. Note, the product may ultimately be lyophilized, powdered, crystalline, liquid tincture or in another physical and/or chemical form.

Resulting extracts may then be orally acceptable compositions applicable to or useful within oral care or oral hygiene products such as toothpaste, mouth rinse, denture adhesive and/or a tooth detergent; particularly as these may be applied into the oral cavity of a corporeal body, and more particularly as these may be applied to an oral feature therein such as on a tooth, a gum, the tongue, on the mucosa, sub-lingually, buccally or labially, or on the hard or soft palate. Such products may be applied in or by a toothbrush or a collapsible swab. Any or all of these may be used as a convenient method of delivery for any nutrient required in substantially small quantities, particularly the phytochemicals described hereinabove, particularly those that can be applied into the body through oral ingestion or trans-mucosal absorption. Oral care products may be especially suited for this purpose when a relatively high level of consumption of a particular supplement on a daily basis may be desirable, particularly for continued reliable ingestion/absorption over the course of many months or years. This is because a vast majority of persons conventionally already have a consistent, almost automatic, daily oral hygiene ritual or habit, regardless of whether that ritual is effective at reducing oral disease. And, while it may be very difficult for many people establish a new habit, as for example, committing to a new daily routine over an extended period (e.g., ten years) that would likely require a significant change in lifestyle (e.g., juicing vegetables and fruits in lieu of meals, daily use of over the counter (OTC) pills or tablets, or every-day consumption of the same or similar foods), it requires virtually no effort at all to obtain consistent delivery of an under-consumed nutrient when such a nutrient is incorporated into a daily-use oral care product. The convenience of delivery, either by unconscious swallowing or direct access to the bloodstream through ingestion by sub-mucosal absorption, makes oral care product delivery mechanisms more efficient and effective that conventional processes.

Moreover, oral care products such as those described here can deliver nutrients through a mechanism of residual product adherence to oral tissues with subsequent release and swallowing of the residual fraction over time. Alternatively and/or additionally, oral care products hereof may further contain ingredients that enhance membrane permeablility and can thereby enhance trans-mucosal absorption, especially sub-lingually. Such ingredients could be selected from a variety of detergents and solvents, used singly or in combination. Furthermore, a tooth cleaning swab containing one or more of solvents, surfactants, chelators, saponifiers, and detergents, and known to enhance rapid penetration, could also simultaneously contain carotenoids or other useful small molecular weight ingredients. Transmucosal absorption may further be enhanced if a delivery device such as a swab is used in a process whereby it may be held under the tongue for a period of, for example, about 2 minutes, particularly if in close proximity to the area where humans have the perhaps the easiest direct access to the blood supply via the sub-lingual mucosal layer that can be as thin as two cells.

Thickeners and/or oral glues can be used in the compositions hereof to increase the amount of residual product retention, at least of a small fraction of the product after typical application and expectoration. Using such additives, as much as about 5% or more of the initially applied amount can adhere onto one or more oral tissues, then release over time, delivering the nutrient to the body through the digestive system or trans-mucosally to the blood stream. Useful thickeners for the purpose of residual product retention herein can be chosen from natural and synthetic gums and colloids such as carrageenan, xanthan gum and sodium carboxymethyl cellulose, as well as gum tragacanth, starch, polyvinylpyrrolidone, hydroxyethyl propylcellulose, hydroxybutyl methyl cellulose, hydroxypropyl methyl cellulose and hydroxyethyl cellulose. Inorganic thickeners, such as colloidal silica, e.g., Syloid 244, and synthetic hectorite, such as Laponite, marketed by Laporte Industries, Ltd, may also or alternatively be used, and mixtures of such thickeners may also be useful. The thickener component of the toothpaste will normally be in the range of about 0.2 to about 5% by weight, preferably from about 1 to about 5% by weight to cause maximum oral retention.

Another class of oral retention agent includes Gantrez S and related copolymers. These orally acceptable glues are substantive to the buccal cavity and teeth, providing a long residence time in the mouth. These polymers coat the teeth and stay attached to the wet buccal surfaces even under conditions of continual saliva secretion. As a result, the polymer's residence time in the mouth was found to be as long as 12 hours. It is possible to vary the length of retention and in essence achieve a time released dosing mechanism when Gantrez S or similar copolymers are used. Although time release dosing is not critical here, the reliable, convenient and effective delivery of other nutrients and pharmaceutical agents that can be delivered through use of the present compositions may increase the desirability of inclusion of such an ingredient.

Although it appears that substantially any oral composition can be used herewith for delivery of the phytochemical or like nutrient, a simple cleaning or oral deodorizing oral care product may be preferred in many implementations to deliver a convenient, reliable (typically every day) dose of an under-consumed supplement or nutrient. In any case, the present compositions do not preclude the co-delivery of other useful oral care benefits which may be considered to be additional active materials or adjuvants. Included in this group may be: other anti-tartar or anti-calculus compounds, such as zinc chloride, zinc acetate and zinc oxide; antibacterial agents such as chlorhexidene, cetyl pyridinium chloride and sanguinaria extract; buffers to control pH; tooth whiteners such as hydrogen peroxide, urea peroxide, sodium perborate and calcium peroxide; preservatives; sweeteners, such as potassium (or sodium) saccharin or cyclamate, acesulfam-K, and aspartame; flavors, such as mint (peppermint or spearmint) and menthol; and dyes or pigments, such as chlorophyll or titanium dioxide.

The present compositions and oral delivery methods may find significant ease of use when formulated as once or twice daily (typically every day) brush-on oral care products such as toothpastes, gels, tooth powders, and liquid dentifrices. An example formulation designed as a brush-on dentifrice delivery composition is presented below. Nevertheless, the benefits hereof can also be delivered through products not brushed onto teeth. Among such products are mouth rinses, antiseptic solutions, chewing gums, tooth treating swabs, such as whitening stain removers in a collapsible swab, plaque locating solutions, and even dental floss and dental tape. In these non-dentifrice preparations the proportion of apple extract and/or carotenoid would typically be greater, due to their lower substantivity, and thus lower inherent ability to adhere a residual amount onto oral tissues. Mouth rinses will normally contain water, alcohol, humectant, such as glycerol, sorbitol and/or polyethylene glycol, flavor and sweetener (typically non-sugar), in addition to the active components mentioned. An example formulation is provided below.

As described in some detailed examples above, the achievement of high carotenoid bio-availability from an apple extract may include disrupting apple cell wall structure so that the cellular contents can be collected. In addition or alternatively to the enzymatic examples above, a pressure and/or temperature method may be used. In this, an extract may be captured in a filtered supernatant after pressing and filtering as described in the exemplar process set forth in the following paragraph.

One (1) pound of dried apple rings (or like fruit or vegetable) may be heated under pressure by submersion in water (enough to cover) in a food grade pressure cooker set at 400° C. for 8 hours. After the heat is removed and the pressure released, the resultant apple compound is brown and decomposed in mash. This process releases the carotenoids contained with the tough cell walls of apple cells. Carotenoids are particularly resistant to decomposition under heat and pressure, and heating using the above method increases the availability, i.e., bio-availability, of beta carotene, vitamin A and lycopene and/or other phytochemical substances derived from apples. The decomposed apple mash may then be pressed through a fine sieve using an apple press, and this yields a brown particulate liquid. The particulate liquid may then be passed through cheese cloth and a paper filter to capture a clear, less colored apple extract liquid. The liquid can be frozen or refrigerated, but preferably frozen and lyophilized into a light white-brown powder which can be more easily stored with less concern about contamination or fermentation. The lyophilized powder can be added directly into oral care compositions or alternatively reconstituted with warm de-ionized water, and then added to oral care compositions as a liquid for added convenience.

Once the extract is prepared, several different compositions of oral care products can be developed to efficiently disperse the bio-available carotenoids within the oral cavity for sublingual absorption or swallowing of any adherent residue formed after normal application of the product. An exemplar implementation of an apple extract containing toothpaste is set forth in the following Table A:

TABLE A
APPLE EXTRACT TOOTHPASTE ( 1 Liter)

| Ingredients | % w/v | Amount in 1 liter | |
|---|---|---|---|
| Vegetable Glycerin | 10.0 | 100 | mL |
| Sorbitol | 20.0 | 200 | mL |
| Hydrated Silica | 20.0 | 200 | mL |
| Purified Water | 30.0 | 300 | mL |
| Xylitol | 20.0 | 200 | mL |
| Carrageenan | 2.0 | 20 | g |
| Sodium Lauryl Sulfate | 2.0 | 20 | g |
| Titanium Dioxide | 2.0 | 20 | g |
| Apple Extract (powder) | 2.0 | 20 | g |
| Apple Flavor Oil | 0.5 | 5 | ml |
| Lycopene or B-carotene (optional) | 0.025 | 0.25 | g |
| FD & C Color | 0.001 | 0.01 | ml |

Such a toothpaste may be prepared by conventional methods of making toothpaste and/or dental creams or dental gels. More specifically, the gelling agent such as a carageenan may be dispersed in vegetable glycerin, to which may be added an aqueous solution containing a sweetening agent such as xylitol, followed by the addition of sorbitol and mixing for a period of about 20 minutes to hydrate the gum, mixing the gum mixture with hydrated silica in a mixer under a vacuum of about 28 to about 30 inches of pressure. Lastly, the apple flavor, the surfactant and optional phytochemical-containing powder (an optional larger amount of phyto-chemical powder, e.g., up to about 50 mg may alternatively be used) may be added to the vacuum mixer, mixed for a period of about 15 minutes, and the final mixture placed in a tube for storage and distribution for end use. In the practice hereof, to increase intake of bio-available carotenoids and simultaneously promote oral hygiene, the toothpaste may be applied regularly to the oral cavity by brushing on the teeth for a period such as 120 or 180 seconds, typically 1 or 2 times a day either after or before meals.

The toothpaste composition hereof has carotenoids and other beneficial phytochemicals from the apple extract, such as one or more vitamins, with sufficiently small molecular size to be absorbed through the lingual mucous membrane of the mouth. The effective additives of the present composition are also water soluble, and may thus ultimately be absorbed into the person's bloodstream to act as a source of bioavailable carotenoids. Furthermore, it may be recommended that the person using such composition avoid rinsing after expectoration to maximize retention. An estimated remaining 2% to 5% residue may adhere onto oral structures to be swallowed over time.

A pink opaque Apple Extract Toothpaste should result with a pleasingly fresh, apple fresh taste (perhaps with cinnamon or mint or other additional flavor) with a pH of about 8.0. The viscosity will be about 150,000 cps. Each 2 g dose should contain between about 25 and about 50 mg carotenoids. A mean residual amount on a hydroxyapatite disc after dipping in saliva and vigorous shaking should be about 5.85%. With such amounts, the mean systemic delivery per use should be between about 2.6 mg and about 5.2 mg per day.

An implementation of a mouthrinse containing a clear apple extract is shown in the following Table B:

TABLE B
APPLE EXTRACT MOUTHRINSE ( 100 Liter)

| | Ingredients | % w/v | Amount in 100 liter | |
|---|---|---|---|---|
| 1. | Sodium Lauryl Sulphate | 0.210 | 210.0 | g. |
| 2. | Benzoic Acid | 0.025 | 25.0 | g. |
| 3. | Sodium Saccharin | 0.050 | 50.0 | g. |
| 4. | Zinc Chloride | 0.005 | 5.0 | g. |
| 5. | Sodium Benzoate | 0.180 | 180.0 | g. |
| 6. | Glycerin | 7.500 | 7.5 | kg. |
| 7. | Vitamin A (beta-carotene) | 0.025 | 25.0 | g. |
| 8. | Apple Flavor Oil | 0.060 | 60.0 | g. |
| 9. | Benzol Alcohol | 0.040 | 40.0 | g. |
| 10. | Oil of Cinnamon | 0.110 | 110.0 | g. |
| 11. | Apple Extract Liquid* | 1.000 | 1.00 | Liter |
| 12. | Polyethylene Glycol | 0.500 | 500.0 | g |
| 13. | Pluronic 105 | 0.500 | 500.0 | g |
| 14. | Deionized Water q.s. to 100.0 L | | | |
| 15. | Benzoic Acid q.s. to adjust pH | | | |
| 16. | Sodium Benzoate q.s to adjust pH | | | |

*reconstituted from adding 10% lyophilized Apple Extract Powder to 90% warm water.

Ten liters of deionized water may be mixed with 7.5 kg of glycerin in a 100 liter container using a high shear mixer. Sodium lauryl sulfate may then be added and mixing continued until all the ingredients are dissolved. Once a clear solution is achieved, the saccharin, zinc chloride and sodium benzoate can be added and mixed until dissolved. The Vitamin A powder can then be added and mixed thoroughly until dissolved.

The flavor oils may be placed in a 500 ml container and mixed until they become uniform. The benzoic acid can then be added and mixed until the solution once again becomes clear. The polyethylene glycol may first be preheated to 40° C. and this then combined in still a third vessel with the poloxamer 105 which may have been pre-heated to 45° C.-60° C. The surfactants may then be added to the essential oil/flavor oils mixture and thoroughly dispersed. Subsequently, the glycerin, sodium lauryl sulfate and buffers combination that was previously mixed can be added to the main batch and mixed for at least about 30 minutes. Water may then be added to q.s. the solution to 100 liters and the pH adjusted, if necessary, using the sodium benzoate or benzoic acid buffers depending on whether the pH needs to be lowered or raised.

A substantially colorless Apple Extract Mouthwash should result with a pleasingly fresh, Cinnamon Apple fresh taste with a pH of about 6.0. The specific gravity should be about 1.021. Each 30 ml dose should contain between about 25 and about 50 mg carotenoids. Mean residual amount on a hydroxyapatite disc after dipping in saliva and vigorous shaking should be about 3.0%. Thus, a mean systemic delivery per use is about 1.2 mg, or about 3.6 mg per day if used 3 times daily.

As a result, it may be noted that such extracts in oral compositions according hereto should provide a desirable supplement of between about 1 and about 6 mg per day (or more if more carotenoid extract is supplied to the overall composition). At the least, a better supplementation above typical levels will result for the user (in many cases, perhaps doubling or even quadrupling the typical amount, e.g., 2 mg). With a minimum diet, the minimum RDA/RDI should be achieved (between about 6 and about 11 mg per day), and with a good diet, or with additional supplementation, an even more effective amount up to the Physician test amount of about 50 mg per day may be achieved. At one or more of these levels, effective health benefits may result. Indeed, supplementation from use of compositions and/or methods hereof may be effective in treatment or in assisting in prevention of a disease such as cardiovascular disease, diabetes, cancer and/or ophthmological disease, e.g., cataracts. These health enhancing agents may further be used in treating elders or others for a variety of physical issues, particularly as these phytochemicals/phytonutrients may contain disease protective anti-oxidant properties. Anti-oxidants of both the enzymatic and non-enzymatic type may be included in the agents and pharmaceutical compositions. The anti-oxidant component, when used, is present in an amount sufficient to inhibit or reduce the effects of systemic free-radicals that can over the long term cause tissue damage, disease and cancer.

Thus disclosed are compositions containing extracts of fruits, vegetables, and/or commonly used leaves or herbs which may have healthful benefits. Also disclosed are methods of use and methods of preparation of such compositions. Of particular note are the oral applications hereof.

The inventive methods for making and/or using the extracts and oral compositions, and the extracts and oral compositions themselves have been described above in considerable detail. This was done for illustrative purposes. Neither the specific implementations of the invention as a whole, nor those of its features, limit the general principles underlying the invention. In particular, the invention is not necessarily limited to the specific constituent materials and proportions of constituent materials used in making the compositions. The invention is also not necessarily limited to oral compositions as specifically composed herein, but extends to other oral care applications as well. The specific features described herein may be used in some implementations, but not in others, without departure from the spirit and scope of the invention as set forth. Many additional modifications are intended in the foregoing disclosure, and it will be appreciated by those of ordinary skill in the art that, in some instances, some features of the invention will be employed in the absence of other features. Additional features may be implemented as well. The illustrative examples therefore do not define the metes and bounds of the invention and the legal protection afforded the invention, which function is served by the claims and their equivalents.

1. A method for delivery of a phytochemical supplement to a corporeal body comprising:
providing a phytochemical supplement in an orally acceptable composition; and,
applying the orally acceptable composition including the phytochemical supplement in contact with a feature of the oral cavity of a corporeal body.

2. A method according to claim 1 further comprising the corporeal body ingesting the phytochemical supplement, or the corporeal body absorbing the corporeal supplement, or both.

3. A method according to claim 1 further comprising the corporeal body ingesting the phytochemical supplement through the digestive system, or absorbing the phytochemical supplement trans-mucosally into the blood stream, or both.

4. A method according to claim 1 wherein the applying operation further includes applying the orally acceptable composition on one or more of a tooth surface, a gum, a tongue, a mucous membrane, a sub-lingual surface, a buccal surface, a labial surface, a soft palate, or a hard palate.

5. (canceled)

6. A method according to claim 1 wherein the applying operation further includes using one or more of a brush and a swab for brushing and swabbing the orally acceptable composition on the feature of the oral cavity.

7. A method according to claim 1 wherein the applying operation further includes using one or more of a brush and a swab for holding the orally acceptable composition in contact with the feature of the oral cavity for a period.

8. (canceled)

9. A method according to claim 1 wherein the applying operation further includes using one or more of a brush and a swab, and the orally acceptable composition is a brush-on or swab-on oral care product of one or more of a toothpaste, gel, tooth powder, and liquid dentifrice.

10. (canceled)

11. (canceled)

12. (canceled)

13. A method according to claim 1 wherein the orally acceptable composition is an oral care mouthrinse product and the applying operation includes rinsing the oral cavity with the mouthrinse.

14. (canceled)

15. A method according to claim 1 wherein the providing operation further includes providing a component for residual product adherence to an oral tissue.

16. (canceled)

17. (canceled)

18. (canceled)

19. (canceled)

20. (canceled)

21. (canceled)

22. An oral care composition comprising:
a phytochemical supplement for one or more of ingestion or trans-mucosal absorption; and,
a carrier.

23. An oral care composition according to claim 22 wherein the phytochemical supplement comprises one or more of a fruit or vegetable extract or an apple extract.

24. An oral care composition according to claim 22 wherein the phytochemical supplement comprises one or more of, a carotenoid, lycopene, or beta-carotene vitamin A.

25. An oral care composition according to claim 22 wherein the phytochemical supplement comprises one or more of a carotenoid, lycopene, beta-carotene vitamin A, Vitamin E (e.g., tocopherol), Vitamin A (retinol), Vitamin C (ascorbic acid), lutein, neoxanthin, violaxanthin, fucoxanthin, canthaxanthin, zeaxanthin, crocetin, capsanthin, cryptoxanthin, astaxanthin, echinacoside, a caffeoyl derivative, a oligomeric proanthocyanidin or proanthanol, grape seed extract, silymarin, milk thistle extract, silybum marianum, ginkgo biloba, and a green tea polyphenol.

26. An oral care composition according to claim 22 wherein the composition is one or more of a toothpaste, gel, tooth powder, liquid dentifrice, denture fixative, denture cleanser, or mouthrinse.

27. An oral care composition according to claim 22 wherein the phytochemical is in an amount from about 1 to about 20 weight percent of the oral care composition.

28. An oral care composition according to claim 22 further containing one or more of solvents, surfactants, chelators, saponifiers, and detergents.

29. An oral care composition according to claim 22 wherein the phytochemical is lyophilized, powdered, crystalline, liquid tincture or liquid form.

30. A method for making an oral care composition comprising:
preparing a phytochemical supplement including applying one or more of heat, pressure and an enzyme to a raw food source to remove a phytochemical from within a cell structure; and,
mixing the phytochemical supplement with a carrier.

31. A method according to claim 30 wherein the enzyme is one or both of pectinase and cellulose.

32. (canceled)

33. (canceled)

34. (canceled)

35. (canceled)

36. (canceled)

37. A method according to claim 30 wherein the phytochemical supplement is one or more of lyophilized, powdered, crystalline, liquid tincture or liquid form.

38. (canceled)

39. (canceled)

40. (canceled)
